(19) United States
(12) Patent Application Publication
(10) Pub. No.: US 2025/0375112 A1
(43) Pub. Date: Dec. 11, 2025

(54) METHOD FOR CUFF-LESS BEAT-TO-BEAT BLOOD PRESSURE ESTIMATION USING TWO RELATIVE BLOOD VOLUME SENSORS ON DIFFERENT APPLIED PRESSURES

(71) Applicant: TALLINN UNIVERSITY OF TECHNOLOGY, Tallinn (EE)

(72) Inventors: Kristjan PILT, Tallinn (EE); Deniss KARAI, Tallinn (EE)

(21) Appl. No.: 19/302,465

(22) Filed: Aug. 18, 2025

Related U.S. Application Data
(62) Division of application No. 17/908,971, filed on Sep. 2, 2022.
(60) Provisional application No. 62/983,789, filed on Mar. 2, 2020.

(51) Int. Cl. A61B 5/02; A61B 5/00

(52) U.S. Cl. CPC A61B 5/02; A61B 5/72

(57) ABSTRACT

The invention describes a measurement method for the continuous non-invasive determination of blood pressure using two blood volume sensors, which are under two different applied pressures. The non-linear function, which is updated for each cardiac cycle, is used to model the relationship between blood pressure and relative blood volume change. The model depends on relative blood volume changes and applied external pressures to the sensors. The derived model needs one point blood pressure calibration. The blood volume sensor can be optical sensor, such as photoplethysmographic sensor, however, any transducer, which converts blood volume or relative blood volume to electrical signal, is applicable. As one possible application, the method can be used for the blood pressure determination at one finger. However, the method is not limited with the blood volume measurement sites (e.g. radial artery etc.).

### CROSS-REFERENCE TO RELATED APPLICATIONS

This application is a divisional of U.S. patent application Ser. No. 17/908,971 filed on Sep. 2, 2022, which is a § 371 national phase filing of International Patent Application No. PCT/IB2021/051740 filed on Mar. 2, 2021, and claims the benefit of U.S. Provisional Patent Application No. 62/983,789 filed on Mar. 2, 2020, the entire contents of which are incorporated herein by reference.

### TECHNICAL FIELD

The present invention relates to a novel method and a device for the non-occlusive continuous non-invasive determination of blood pressure using two blood volume sensors, which are under two different applied pressures. More specifically, the present invention relates to use of a non-linear function, which is newly updated for every cardiac cycle, to model the relationship between blood pressure and relative blood volume change.

### BACKGROUND OF THE INVENTION

The method proposed by J. Penaz's as so-called “volume-clamp” method as a possibility for continuous recording of blood pressure has been further developed by several authors. The common disadvantage of all devices operating on the “volume-clamp” principle is that a) the device requires a servo system which is expensive and technically complex and cumbersome and b) the operating point needs frequent adjustment.

Devices for measuring the continuous arterial blood pressure of a finger are known, these devices are recording a volume change curve (for example a photoplethysmogram) and calculating a pressure curve from it.

Patent document U.S. Pat. No. 5,296,310, Jones et al., 14 Dec. 1993 describes a method in which the systolic and diastolic pressure values for each cardiac cycle are obtained from the volume curve by multiplying the latter by a constant k. The method is inaccurate because the pressure and volume curves are not linearly related.

U.S. Pat. No. 4,846,189, Sun Shuxing, 11.07.1989 and U.S. Pat. No. 5,423,322, Clark et al., 13 Jun. 1995 assume that the relationship between pressure and volume curves is exponential. This gives a more accurate result in the calculations, but is still inaccurate, because the dependence of the function between the pressure and volume curves changes over time depending on the physiological condition of the person.

### SUMMARY OF INVENTION

The present invention provides a method and apparatus for blood pressure measurement in the non-occlusive non-invasive continuous manner. The device comprises two optical, for example photoplethysmographic, sensors arranged side by side. The optical sensor consists of a light emitting diode and a photodiode that are placed next to each other at determined distance. The optical sensors are under two different applied pressures, which is realized with the cavity in the housing of the device. The surface of first optical sensor in relation to the second optical sensor is placed in the cavity. Both optical sensors are equipped with force transducer that measures the pressure that is applied by the optical sensor to the artery or microvascular bed of tissue. Alternatively, in order to produce differences in the back pressures exerted by the optical sensor, a spring is attached between the first optical sensor and the force transducer, the stiffness of which differs from that of the spring attached between the second optical sensor and the force transducer. The output voltage is in known relation with the applied force on the transducer. The LED of the optical sensor emits light that is absorbed and scattered in the artery or microvascular bed of tissue and fraction of photons are detected by photodiode. The detected pulsatile light intensity changes are related to the relative blood volume changes in the artery or microvascular bed of tissue. The photodiode signals from the optical sensors are connected to transimpedance amplifiers that convert the photocurrents of the photodiodes to the voltage signals. Voltage signals from the force transducers and transimpedance amplifiers are supplied to analogue-to-digital converter (ADC). The digital signals from ADC are supplied to microcontroller, where the volume difference signal amplitude ΔV₁₂ or ΔV₂₁ is calculated based on the signals from optical sensors. In addition, the cardiac cycles are detected and for each cycle the arterial compliance index k is calculated based on the relative blood volume change signals from the optical sensors and the pressures that are applied by the optical sensors. Memory is connected to the microcontroller, which is used to store the calibration parameter and signals during calibration manoeuvre. In addition, during the calibration manoeuvre the systolic and diastolic blood pressures are possible to supply to the microcontroller via external communication port, e.g. USB, Bluetooth etc., that is connected to microcontroller.

The above described device is firstly calibrated to determine certain parameter that is used by the microcontroller to continuously measure the systolic blood pressure (SBP), diastolic blood pressure (DBP), and pulse pressure (PP). There are two possible calibration manoeuvres. The first possible calibration manoeuvre includes the external device that determines the arterial blood pressure, e.g. oscillometric blood pressure device. The arterial blood pressure is measured by external blood pressure device and at the same time the calibration manoeuvre is initiated in the device via external communication port. During the calibration manoeuvre amplitudes ΔV₁₂ or ΔV₂₁ of the relative volume change differences, parameter k, and applied pressure signals are recorded to the memory. The recording is terminated in the microcontroller via external communication port after the blood pressure measurement is finished with the external device. As follows, the systolic blood pressure and diastolic blood pressure are supplied to the microcontroller via external communication port. The calibration parameter B is calculated based on the recorded data and blood pressure values.

The second possible calibration manoeuvre is initiated, when the microcontroller detects the rise in the force that is applied to the optical sensors or initiated via external communication port. The volume difference signal amplitude ΔV₁₂ or ΔV₂₁, arterial compliance index k, and applied pressure signal values are recorded to the memory for each cardiac cycle. The applied forces on the optical detectors can be monitored via external communication port. The applied pressure by the optical sensors is increased (e.g. manually with finger) and it exceeds the mean arterial blood pressure. Thereafter, the applied pressure is decreased back to the initial level, which is detected by the microcontroller, and the recording of the parameters to the memory is terminated automatically or via external communication port. The maximal values ΔV_{12_max} or ΔV_{21_max} of amplitudes ΔV₁₂ or ΔV₂₁ from the recorded time series is detected. Based on this point in the time series, the arterial compliance index kₘₐₓ and pressure sensor values P_{s1_max} and P_{s2_max} are detected and calibration parameter B is calculated.

The function (compliance model) between blood pressure and relative blood volume change is determined based on the calibration parameter B for particular patient and for every cardiac cycle updated compliance index k. The calculated systolic blood pressure, diastolic blood pressure, and pulse pressure values in the microcontroller are supplied via external communication port.

### BRIEF DESCRIPTION OF DRAWINGS

The present invention will be described below in detailed description with reference to the accompanied drawings where:

FIG. 1 shows the relationship between transmural pressure and blood volume in artery;

FIG. 2 shows the relationship between transmural pressure and compliance of artery;

FIG. 3 shows a blood volume change in artery in case mean transmural pressure is zero;

FIG. 4 shows a blood volume changes in artery for two pressures sensors at different applied pressures;

FIG. 5 shows a blood volume change in artery between two pressures sensors at different applied pressures;

FIG. 6 illustrates a block diagram of a non-occlusive and continuous blood pressure sensor device, which is constructed according to the principles of current invention;

FIG. 7 illustrates a construction principles of the blood pressure sensor device; a) is a cross section of the pressure sensor device, b) is a bottom view of the pressure sensor device;

FIG. 8 illustrates a construction principles of an alternative solution of blood pressure sensor device; a) is a cross section of the pressure sensor device, b) is a bottom view of the pressure sensor device;

FIG. 9 illustrates a flowchart of blood pressure monitoring and first calibration manoeuvre;

FIG. 10 illustrates a flowchart of blood pressure monitoring and second calibration manoeuvre;

FIG. 11 illustrates calculated volumes ΔV₁, ΔV₂, and ΔV₂₁;

FIG. 12 illustrates applied pressures on optical sensors;

FIGS. 13 to 16 illustrates results of estimated blood pressures using equations according to invention;

FIGS. 17 to 20 illustrates the Bland-Altman plots of the results illustrated in FIGS. 13 to 16.

### DETAILED DESCRIPTION OF THE INVENTION

The present invention provides for non-occlusive non-invasive continuous imposed arterial blood pressure monitoring. The systolic blood pressure, diastolic blood pressure and pulse pressure are obtained by calculation using arterial blood volume signals from two volume sensors, which are under two different applied pressures. The volume signals are obtained optically using optical sensing technique, which is widely known, and they represent the relative blood volume changes over time. The arterial blood pressure is estimated using the function, which relates the transmural pressure and compliance in the artery, and it is updated for each cardiac cycle. The function is based on the so-called compliance model, which has been discussed earlier in Baker, P. D., Westenskow, D. R. and Kück, K., “Theoretical analysis of non-invasive oscillometric maximum amplitude algorithm for estimating mean blood pressure”, Med. Biol. Eng. Comput. 35, 1997, page 271-278.

Transmural pressure Pₜ is the difference between the intra-arterial pressure P and the externally applied pressure Pₛ (e.g. applied by optical sensor). Transmural pressure is calculated as follows:

$${P}_{t} = P - {P}_{s} \qquad ( 1 )$$

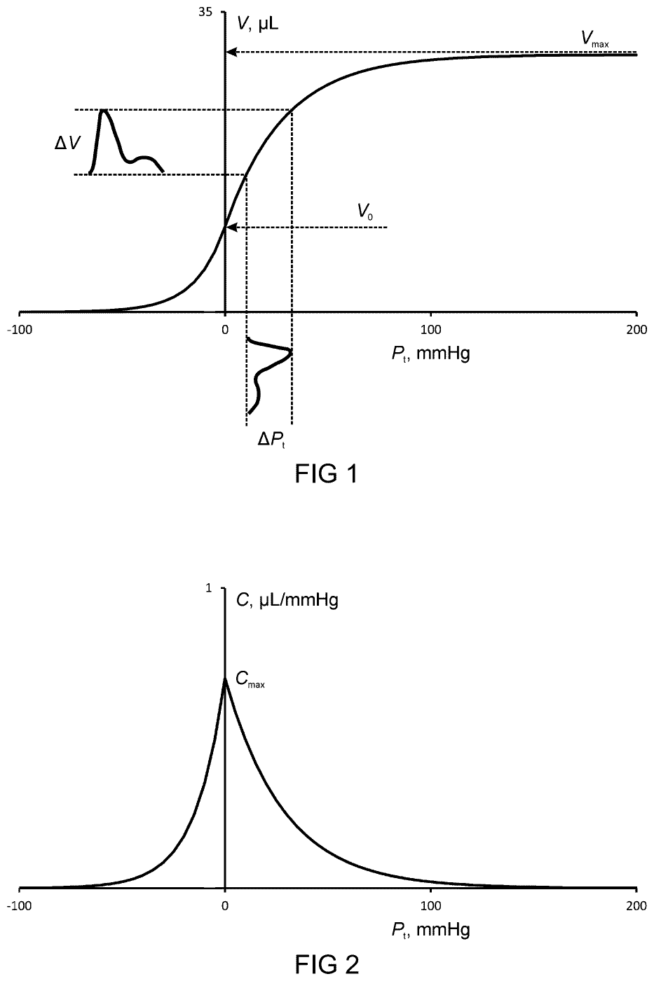

The blood volume V in artery and transmural pressure are related to each other through relationship, which is given in FIG. 1. The blood volume in artery is given with the following equation, in case the Pₜ>0:

$$V = {V}_{max} - ( {V}_{max} - {V}_{0} ) \cdot {e}^{- \frac{{c}_{m}}{( {V}_{max} - {V}_{0} )} \cdot {P}_{t}} \qquad ( 2 )$$

where Vₘₐₓ is the is the maximum arterial volume when the artery is fully expanded, V₀ is the arterial volume at zero Pₜ, and Cₘ is the maximum compliance. It can be seen that even with the same change of transmural pressure ΔPₜ the volume change ΔV is different depending on the operating point of Pₜ (FIG. 1). ΔV represents the relative volume change or amplitude within one cardiac cycle.

Through differentiation of equation 9 the analytical form can be obtained for the arterial compliance, in case Pₜ>0:

$$C = \frac{d V}{d {P}_{t}} \approx \frac{Δ V}{Δ {P}_{t}} = {C}_{m} \cdot {e}^{- \frac{{c}_{m}}{( {V}_{max} - {V}_{0} )} \cdot {P}_{t}} \qquad ( 3 )$$

The relationship is illustrated in FIG. 2.

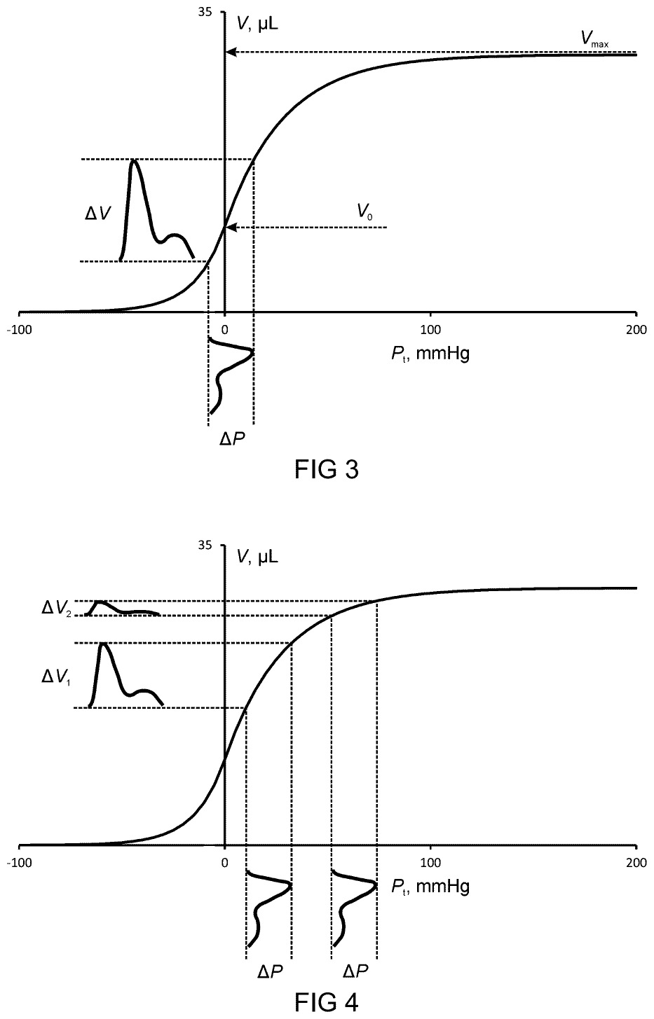

Blood volume change in artery is maximal in case mean transmural pressure is zero (see FIG. 3). In such case the externally applied pressure is equal to the mean arterial pressure.

In the non-occlusive continuous (beat-to-beat) blood pressure estimation system the two blood volume sensors, S1 and S2, which are optical sensors in the present invention, are applied to the artery at two different pressures Pₛ₁ and Pₛ₂. In such case the blood pressure change ΔP in the artery is equal to the pulse pressure. For both blood volume sensors, the pulse pressure is the same; however, the blood volume changes under the sensor are different. The blood volume change for volume sensor with applied pressure Pₛ₁ is equal to ΔV₁ and for volume sensor with applied pressure Pₛ₂ is equal to ΔV₂.

For both volume sensors, the compliances of artery can be calculated as follows:

$${C}_{1} = \frac{Δ {V}_{1}}{Δ P} \qquad ( 4 )$$

$${C}_{2} = \frac{Δ {V}_{2}}{Δ P}$$

As pulse pressures are equal for both sensors (assuming that pulse pressure is not changing in such a short distance between two sensors) then from equation 4:

$$\frac{{C}_{1}}{{C}_{2}} = \frac{Δ {V}_{1}}{Δ {V}_{2}} \qquad ( 5 )$$

By substituting equation 3 to equation 5:

$$\frac{Δ {V}_{1}}{Δ {V}_{2}} = \frac{{C}_{m} \cdot {e}^{- \frac{{C}_{m}}{( {V}_{max} - {V}_{0} )} \cdot {P}_{t 1}}}{{C}_{m} \cdot {e}^{- \frac{{C}_{m}}{( {V}_{max} - {V}_{0} )} \cdot {P}_{t 2}}} = {e}^{- \frac{{C}_{m}}{( {V}_{max} - {V}_{0} )} \cdot ( {P}_{t 1} - {P}_{t 2} )} \qquad ( 6 )$$

$$\frac{ln ( \frac{Δ {V}_{1}}{Δ {V}_{2}} )}{( {P}_{t 1} - {P}_{t 2} )} = \frac{{C}_{m}}{( {V}_{max} - {V}_{0} )} = k . \qquad ( 7 )$$

The equation 5 can be represented as well with opposite ratios:

$$\frac{{C}_{2}}{{C}_{1}} = \frac{Δ {V}_{2}}{Δ {V}_{1}} . \qquad ( 8 )$$

By substituting equation 3 to equation 8:

$$\frac{ln ( \frac{Δ {V}_{2}}{Δ {V}_{1}} )}{( {P}_{t 2} - {P}_{t 1} )} = \frac{{C}_{m}}{( {V}_{max} - {V}_{0} )} = k . \qquad ( 9 )$$

The difference between transmural pressures of Pₜ₁ and Pₜ₂ (Pₜ₁<Pₜ₂) is equal to the difference between applied pressures of volume sensors Pₛ₁ and Pₛ₂ (Pₛ₁>Pₛ₂), which can be calculated as follows:

$${P}_{t 1} = P - {P}_{s 1} , \qquad ( 10 )$$

$${P}_{t 2} = P - {P}_{s 2} , \qquad ( 11 )$$

$${P}_{t 1} - {P}_{t 2} = P - {P}_{s 1} - P + {P}_{s 2} = {P}_{s 2} - {P}_{s 1} and \qquad ( 12 )$$

$${P}_{t 2} - {P}_{t 1} = P - {P}_{s 2} - P + {P}_{s 1} = {P}_{s 1} - {P}_{s 2} . \qquad ( 13 )$$

Therefore, the equations 7 and 9 can be rewritten as follows:

$$\frac{ln ( \frac{Δ {V}_{1}}{Δ {V}_{2}} )}{( {P}_{s 2} - {P}_{s 1} )} = \frac{{C}_{m}}{( {V}_{max} - {V}_{0} )} = k and \qquad ( 14 )$$

$$\frac{ln ( \frac{Δ {V}_{2}}{Δ {V}_{1}} )}{( {P}_{s 1} - {P}_{s2} )} = \frac{{C}_{m}}{( {V}_{max} - {V}_{0} )} = k . \qquad ( 15 )$$

The compliance model in equation 3 can be rewritten based on the equations 14 and 15:

$$C = k \cdot ( {V}_{max} - {V}_{0} ) \cdot {e}^{- k \cdot {P}_{t}} . \qquad ( 16 )$$

By knowing the difference between applied pressures of volume sensors and estimated relative blood volume changes the k can be calculated using equations 14 or 15 and it is dependent on compliance of artery. It is known that the compliance of artery changes due to the slowly varying tonus of the muscles around the vessel driven by the nervous system. Therefore, the calculation of parameter k for each cardiac cycle updates the compliance model. It is assumed that the difference (Vₘₐₓ−V₀) is not changing because maximal volume of artery cannot increase or decrease (during short period of time the artery is not growing bigger) and can be estimated by individual calibration. Therefore, in the following text the difference (Vₘₐₓ−V₀) is substituted by calibration parameter B.

The difference between transmural pressures is equal to the difference between applied pressures of volume sensors:

$$Δ {P}_{s 12} = {P}_{s 1} - {P}_{s 2} or \qquad ( 17 )$$

$$Δ {P}_{s 21} = {P}_{s 2} - {P}_{s 1} . \qquad ( 18 )$$

The difference between applied pressures of volume sensors corresponds to the measured blood volume difference by volume sensor signals V₁ and V₂, and can be calculated as follows:

$${V}_{12} = {V}_{1} - {V}_{2} or \qquad ( 19 )$$

$${V}_{21} = {V}_{2} - {V}_{1} . \qquad ( 20 )$$

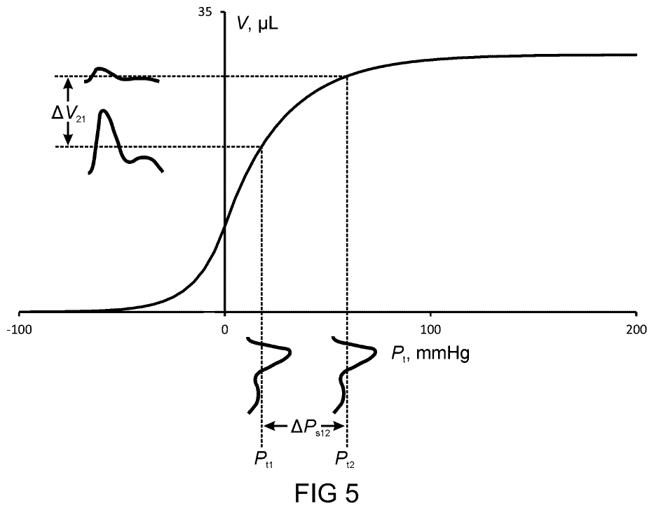

The amplitudes ΔV₁₂ or ΔV₂₁ of the volume difference signals V₁₂ or V₂₁ are detected for every cardiac cycle, respectively, and illustrated in FIG. 5.

In such case, the compliance can be calculated based on equations 4 and 16 for the transmural pressure Pₜ₁+0.5·ΔPₛ₁₂ as follows, in case Pₜ>0:

$$\frac{{ΔV}_{21}}{Δ {P}_{s 12}} = k \cdot B \cdot {e}^{- k \cdot ( {P}_{t 1} + 0.5 \cdot {ΔP}_{s 12} )} . \qquad ( 21 )$$

By substituting equation 1 into equation 21 it can be rewritten:

$$\frac{{ΔV}_{21}}{{ΔP}_{s 12}} = k \cdot B \cdot {e}^{- k \cdot ( P - {P}_{s 1} + 0.5 \cdot {ΔP}_{s 12} )} . \qquad ( 22 )$$

The intra-arterial pressure P derives from the equation 22 as follows:

$$P = {P}_{s 1} - 0.5 \cdot Δ \cdot {P}_{s 12} - \frac{1}{k} \cdot ln ( \frac{{ΔV}_{21}}{Δ {P}_{s 12} \cdot k \cdot B} ) . \qquad ( 23 )$$

Similarly, to the equation 21, the compliance model can be rewritten for the amplitude ΔV₁₂:

$$\frac{{ΔV}_{12}}{Δ {P}_{s 21}} = k \cdot B \cdot {e}^{- k \cdot ( {P}_{t 1} + 0.5 \cdot {ΔP}_{s 12} )} . \qquad ( 24 )$$

In such case the amplitude ΔV₁₂ and difference between applied pressures of volume sensors ΔPₛ₂₁ are both negative. Based on the equation 1 and 24 the P derives similarly to the equation 23 as follows:

$$P = {P}_{s 1} - 0.5 \cdot {ΔP}_{s 12} - \frac{1}{k} \cdot ln ( \frac{Δ {V}_{12}}{Δ {P}_{s 21} k \cdot B} ) . \qquad ( 25 )$$

The P can be also derived from the equations 23 and 25 for the transmural pressure Pₜ₂−0.5. ΔPₛ₁₂ as follows, in case Pₜ>0:

$$P = {P}_{s 2} + 0.5 \cdot Δ {P}_{s 12} - \frac{1}{k} \cdot ln ( \frac{Δ {V}_{21}}{Δ {P}_{s 12} \cdot k \cdot B} ) , and \qquad ( 26 )$$

$$P = {P}_{s 2} + 0.5 \cdot Δ {P}_{s 12} - \frac{1}{k} \cdot ln ( \frac{Δ {V}_{12}}{Δ {P}_{s 21} \cdot k \cdot B} ) . \qquad ( 27 )$$

The equations 23, 25, 26, and 27 can be obtained respectively for the transmural pressures Pₜ₁−0.5·ΔPₛ₂₁ and Pₜ₂+0.5·ΔPₛ₂₁, in case Pₜ>0:

$$P = {P}_{s 1} + 0.5 \cdot Δ {P}_{s 21} - \frac{1}{k} \cdot ln ( \frac{Δ {V}_{21}}{Δ {P}_{s 12} \cdot k \cdot B} ) \qquad ( 28 )$$

$$P = {P}_{s 1} + 0.5 \cdot Δ {P}_{s 21} - \frac{1}{k} \cdot ln ( \frac{Δ {V}_{12}}{Δ {P}_{s 21} \cdot k \cdot B} ) \qquad ( 29 )$$

$$P = {P}_{s 2} - 0.5 \cdot Δ {P}_{s 21} - \frac{1}{k} \cdot ln ( \frac{Δ {V}_{21}}{Δ {P}_{s 12} \cdot k \cdot B} ) \qquad ( 30 )$$

$$P = {P}_{s 2} - 0.5 \cdot Δ {P}_{s 21} - \frac{1}{k} \cdot ln ( \frac{Δ {V}_{12}}{Δ {P}_{s 21} \cdot k \cdot B} ) \qquad ( 31 )$$

Intra-arterial pressure P can be estimated equally from equations 23, 25, 26, 27, 28, 29, 30, and 31, in case the calibration parameter B is determined through one point calibration. Therefore, the B is calculated in case the intra-arterial pressure P is known and it is derived from the equations 23 and 25:

$$B = \frac{Δ {V}_{21}}{( {P}_{s 1} - {P}_{s 2} ) \cdot k \cdot {e}^{- k \cdot ( P - {P}_{s 1} + 0.5 \cdot ( {P}_{s 1} - {P}_{s 2} ) )}} , \qquad ( 32 )$$

$$B = \frac{Δ {V}_{12}}{( {P}_{s 2} - {P}_{s 1} ) \cdot k \cdot {e}^{- k \cdot ( P - {P}_{s 1} + 0.5 \cdot ( {P}_{s 1} - {P}_{s 2} ) )}} . \qquad ( 33 )$$

Similarly, the calibration parameter B can be derived from all the intra-arterial pressure P equations 26 to 31. However, in the following text all the derivations are based on the equations 23 and 25. The intra-arterial pressure P can be determined using for example an external oscillometric blood pressure measurement device and the systolic blood pressure (SBPₘ), diastolic blood pressure (DBPₘ), mean blood pressure (MBPₘ), and pulse pressure (PPₘ) are measured. Any of previously mentioned two measured blood pressures can be selected for the intra-arterial pressure P calculation as they are all related to each other. However, here the intra-arterial pressure P is calculated using systolic and diastolic blood pressure and the calibration parameter B derives as follows based on the equations 32 and 33:

$$B = \frac{Δ {V}_{21 \_ m}}{\begin{matrix}( {P}_{s 1 \_ m} - {P}_{s2\_ m} ) \cdot {k}_{m} \cdot {e}^{- {k}_{m}} \cdot \\ {( {DBP}_{m} + 0.5 \cdot ( {SBP}_{m} - {DBP}_{m} ) -}^{} \\ {{P}_{s 1 \_ m} + 0.5 \cdot ( {P}_{s 1 \_ m} - {P}_{s 2 \_ m} )}^{} )\end{matrix}} , and \qquad ( 34 )$$

$$B = \frac{Δ {V}_{12 \_ m}}{\begin{matrix}\begin{matrix}( {P}_{s 2 \_ m} - {P}_{s1\_ m} ) \cdot {k}_{m} \cdot {e}^{- {k}_{m}} \cdot \\ {}^{( {DBP}_{m} + 0.5 \cdot ( {SBP}_{m} - {DBP}_{m} ) -}\end{matrix} \\ {{P}_{s 1 \_ m} + 0.5 \cdot ( {P}_{s 1 \_ m} - {P}_{s 2 \_ m} )}^{} )\end{matrix}} , \qquad ( 35 )$$

where ΔV_{21_m}, kₘ, P_{s1_m}, P_{s2_m} are the average values of parameters ΔV₂₁, ΔV₁₂, k, Pₛ₁, Pₛ₂ during the period while blood pressure measurement was carried out by external device. The calibration parameter B is derived as well in case the transmural pressure is zero (P−Pₛ₁+0.5·ΔPₛ₁₂=0) in equation 22. In such case the amplitudes ΔV₁₂ or ΔV₂₁ of the volume difference signals are maximal ΔV₁₂ max or ΔV₂₁ max. This situation is achieved by increasing the pressure, which is applied on volume sensors. The calibration parameter B is derived from the equation 22 for ΔV_{12_max} or ΔV_{21_max}:

$$B = \frac{Δ {V}_{21 \_ max}}{( {P}_{s 1 \_ max} - {P}_{s 2 \_ max} ) \cdot {k}_{max}} or \qquad ( 34 )$$

$$B = \frac{Δ {V}_{12 \_ max}}{( {P}_{s 2 \_ max} - {P}_{s1\_ max} ) \cdot {k}_{max}} , \qquad ( 35 )$$

where kₘₐₓ, P_{s1_max} and P_{s2_max} are the values of k, Pₛ₁, and Pₛ₂ at the situation when ΔV₁₂ or ΔV₂₁ are maximal.

The compliance model is used for the intra-arterial pressure P calculations once the calibration parameter B is estimated. Based on the calculated intra-arterial pressure P, the pulse pressure (PP) is calculated by combining equations 4, 10, 11, and 16:

$$P P = \frac{Δ {V}_{1}}{k \cdot B \cdot {e}^{- k \cdot ( P - {P}_{s 1} )}} , \qquad ( 36 )$$

$$PP = \frac{Δ {V}_{2}}{k \cdot B \cdot {e}^{- k \cdot ( P - {P}_{s 2} )}} , \qquad ( 37 )$$

Systolic blood pressure is calculated based on equations 23, 36, and 37 as follows:

$$SBP = P + 0.5 \cdot PP . \qquad ( 38 )$$

Similarly, diastolic blood pressure is calculated based on equations 23, 36 and 37 as follows:

$$DBP = P - 0.5 \cdot PP . \qquad ( 39 )$$

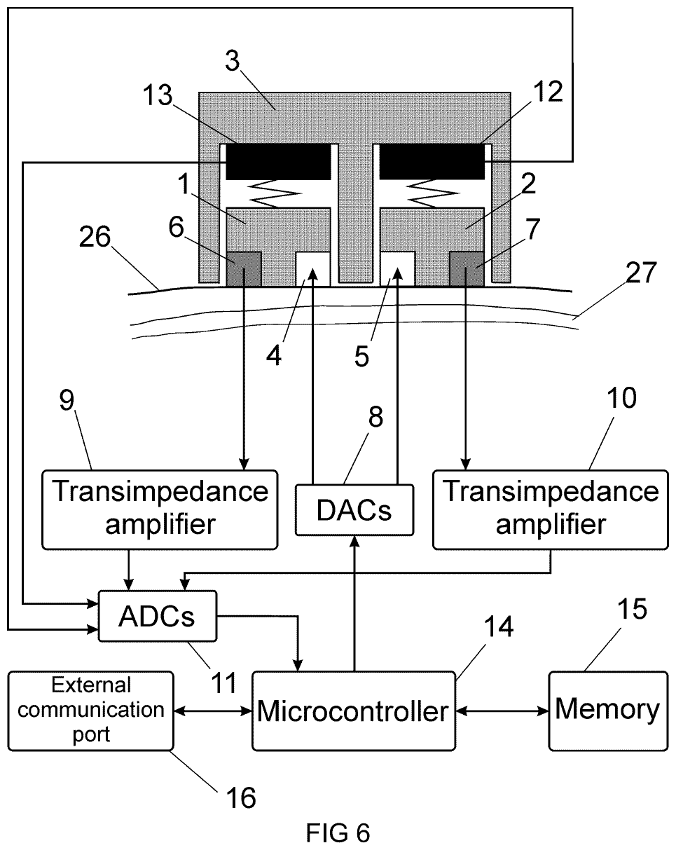

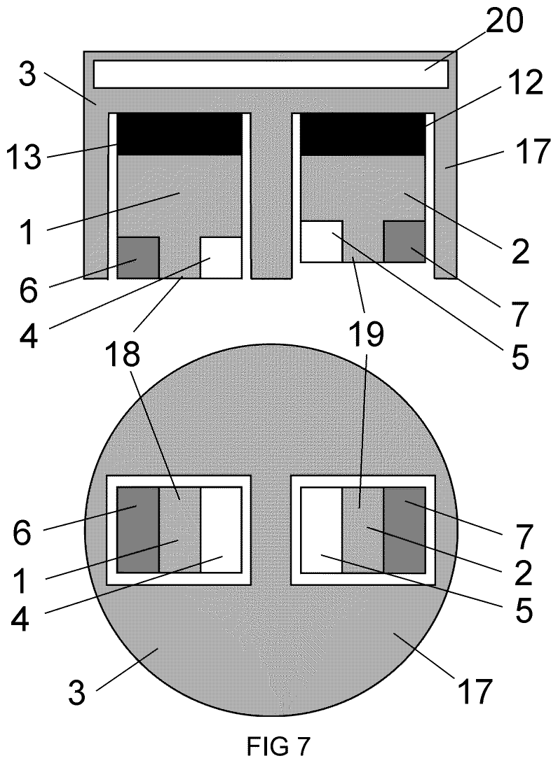

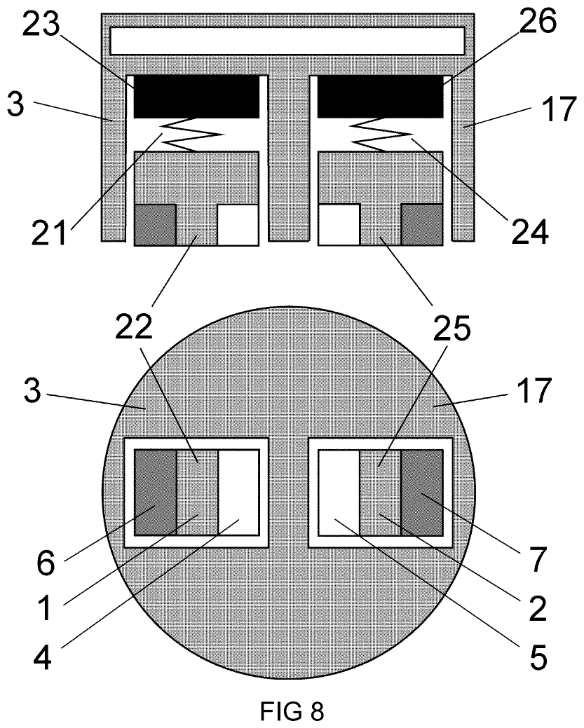

In the present invention, the device for non-occlusive non-invasive continuous pressure monitoring is shown in FIG. 6. The dependence between the transmural pressure and compliance for each cardiac cycle is performed by updating the function (compliance model) adopted for sensor device. The sensor device comprises two pairs of photoplethysmographic sensors 1, 2 arranged side by side in one sensor device housing 3 as optical sensor comprising of a light source 4, 5 and a photodetector 6, 7, digital-to-analogue converters (DACs) 8 electrically connected to light source, transimpedance amplifiers 9, 10 electrically connected to the photodetector, analogue-to-digital converters (ADCs) 11 electrically connected to the force transducers 12, 13 and the transimpedance amplifiers, a microcontroller 14 electrically connected to the analogue-to-digital and digital-to-analogue converters, an electrically connected memory 15 and external communication port 16 to the microcontroller. A force transducer is attached to each optical sensor. The back pressure exerted on the artery by both optical sensors can be measured with a force transducer. The sensor housing 17, shown in FIG. 7, comprises one or both optical sensors 18, 19 in the case of cavities. The sensor housing is designed so that the surfaces of the optical sensors are not in the same level. The surface of one optical sensor is located in relation to the surface of the other sensor in the cavity. Thereby, the optical sensor in the cavity exerts a lower back pressure than the sensor not in the cavity. The optical sensor and force transducer in the cavity is attached to the housing so that the arterial pressure exerted by the optical sensor can be recorded. The signals from the optical sensors and force transducers are supplied to the other electrical components of device 20. In alternative embodiment, shown in FIG. 8, in order to produce differences in the back pressures exerted by the optical sensors, a first spring 21 is attached between the first optical sensor 22 and the force transducer 23, the stiffness of which differs from that of the second spring 24 attached between the second optical sensor 25 and the force transducer 26.

The light from light emitting diodes (LEDs) is absorbed and scattered in the artery or microvascular bed of tissue and fraction of photons are detected by photodiode (photodetector). The current signal from photodiodes of optical sensors are supplied to transimpedance amplifiers that convert the photocurrents of the photodiodes to the voltage signals. The back pressure exerted on the artery by both optical sensors is measured with a force transducer. The output voltage of the transducer is in known relation with the applied force on the transducer. The outputs of the two transimpedance amplifiers and force transducers are supplied to the analogue-to-digital converters, where the signals are digitized for application to the microcontroller.

The microcontroller turns the LEDs on alternately through the DAC and the intensity of the LEDs are set based on the received voltage signals of photodetectors from the transimpedance amplifier. The driving frequency of the LEDs is at least 1 kHz and the duty cycle is between 25% to 50%. The microcontroller assembles the light intensity signals based on the voltage signals received for each photodetector, while the LED is turned on. Microcontroller may cancel the ambient light by using the voltage signal while the LED is turned off and subtracting it from the signal while the LED is turned on. The relative volume signals V₁ and V₂ are computed using the principles of Beer-Lamber law:

$$I = {I}_{0} \cdot {e}^{- μ \cdot V} , \qquad ( 40 )$$

where I₀ is emitted light intensity by LED, I is detected light intensity by photodiode, V is tissue volume and μ is absorption. In diastole, the arterial blood volume in tissue is minimal Vₘᵢₙ and the detected light intensity is maximal Iₘₐₓ. Beer-Lambert law is as follows:

$${I}_{max} = {I}_{0} \cdot {e}^{- μ \cdot {V}_{min}} \qquad ( 41 )$$

In systole, the arterial blood volume in tissue is maximal and the detected light intensity is minimal. For such case the Beer-Lambert law is as follows:

$${I}_{min} = {I}_{0} \cdot {e}^{- μ ⋅ {V}_{max}} . \qquad ( 42 )$$

Therefore, the relative blood volume change in tissue is:

$$\frac{{I}_{max}}{{I}_{min}} = \frac{{I}_{o} \cdot {e}^{- μ \cdot V} min}{{I}_{o} {e}^{- μ \cdot V} max} = \frac{{e}^{- μ \cdot V} min}{{e}^{- μ \cdot V} max} = {e}^{- μ \cdot {V}_{min} + μ \cdot {V}_{max}} \qquad ( 43 )$$

$$ln ( \frac{{I}_{min}}{{I}_{max}} ) = - μ \cdot {V}_{min} + μ \cdot {V}_{max} = {V}_{max} - {V}_{min} = Δ V .$$

Microcontroller detects for each cardiac cycle the minimal and maximal values of light intensities for both sensors and calculates volume changes ΔV₁ and ΔV₂ using the equation 43.

For the optical sensor S1 the relative blood volume can be calculated as follows:

$${I}_{1} = {I}_{01} \cdot {e}^{- μ \cdot {V}_{1}} \qquad ( 44 )$$

where I₀₁ is the emitted and I₁ is detected light intensity of optical sensor S1. Similarly, the light intensity can be calculated for the second optical sensor S2:

$${I}_{2} = {I}_{02} \cdot {e}^{- μ \cdot {V}_{2}} . \qquad ( 45 )$$

The difference between blood volumes underneath the sensors are calculated as follows:

$$\frac{{I}_{1}}{{I}_{2}} = \frac{{I}_{01} \cdot {e}^{- μ \cdot {V}_{1}}}{{I}_{02} \cdot {e}^{- μ \cdot {V}_{2}}} = \frac{{I}_{1}}{{I}_{2}} \cdot {e}^{- μ \cdot {V}_{1} + μ \cdot {V}_{2}}$$

$$ln ( \frac{{I}_{1} \cdot {I}_{02}}{{I}_{2} \cdot {I}_{01}} ) = {V}_{2} - {V}_{1} = {V}_{21} or \qquad ( 46 )$$

$$ln ( \frac{{I}_{2} \cdot {I}_{01}}{{I}_{1} \cdot {I}_{02}} ) = {V}_{1} - {V}_{2} = {V}_{12} . \qquad ( 47 )$$

Microcontroller calculates according to the equation 46 or 47 the difference between blood volumes underneath the optical sensors and detects the amplitude ΔV₂₁ or ΔV₁₂ for each cardiac cycle, respectively. Furthermore, microcontroller calculates for each cardiac cycle pressures of the sensors Pₛ₁ and Pₛ₂ using the output voltages from force transducers, volume changes ΔV₁ and ΔV₂, parameter k (compliance index), intra-arterial blood pressure P, pulse pressure PP, systolic blood pressure SBP, and diastolic blood pressure DBP, and supplies the values together with parameters ΔV₂₁ or ΔV₁₂ via external communication port.

During calibration procedure the microcontroller stores the parameters ΔV₂₁ or ΔV₁₂, k, Pₛ₁, Pₛ₂, for each cardiac cycle to the memory of the device. There is possibility to initiate and to terminate the calibration manoeuvre via external communication port. The parameter B is calculated and stored to the memory of the device after calibration manoeuvre by microcontroller.

Use of blood pressure monitoring device will now be described. The device is placed on surface of the skin 26 above the subject's artery 27 or microvascular bed of tissue under interest (FIG. 6). An external force is applied to the device, which may be exerted, for example, by a strap attached around the device and the body to be examined. Firstly, the calibration parameter B is determined through calibration manoeuvre. There are two possible calibration manoeuvres.

The first possible calibration manoeuvre includes the external device that determines the arterial blood pressure, e.g. oscillometric blood pressure device. The arterial blood pressure is measured by external blood pressure device and at the same time the calibration manoeuvre is initiated in the device via external communication port. During the calibration manoeuvre amplitudes ΔV₁₂ or ΔV₂₁ of the relative volume change differences, parameter k, and applied pressure signals Pₛ₁ and Pₛ₂ are recorded to the memory. The recording is terminated in the microcontroller via external communication port after the blood pressure measurement is finished with the external device. As follows, the systolic blood pressure (SBPₘ) and diastolic blood pressure (DBPₘ) are supplied to the microcontroller via external communication port. The calibration parameter B is calculated based on the average values of the recorded parameters ΔV₁₂ or ΔV₂₁, parameter k, and applied pressure signals Pₛ₁ and Pₛ₂, and blood pressure values SBPₘ and DBPₘ.

The second possible calibration manoeuvre is initiated, when the microcontroller detects the rise in the force that is applied to the optical sensors or initiated via external communication port. The volume difference signal amplitude ΔV₁₂, arterial compliance index k, and applied pressure signal values Pₛ₁ and Pₛ₂ are recorded to the memory for each cardiac cycle. The applied forces on the optical detectors can be monitored via external communication port. The applied pressure by the optical sensors is increased (e.g. manually) and it exceeds the mean arterial blood pressure. Thereafter, the applied pressure is decreased back to the initial level, which is detected by the microcontroller, and the recording of the parameters to the memory is terminated automatically or via external communication port. The maximal values ΔV_{12_max} or ΔV_{21_max} of amplitudes ΔV₁₂ or ΔV₂₁ from the recorded time series is detected. Based on this point in the time series, the arterial compliance index kₘₐₓ and pressure sensor values P_{s1_max} and P_{s2_max} are detected and calibration parameter B is calculated.

Possible recalibration may be needed periodically depending on the time period that the device has been used continuously.

After calibration the function (compliance model) between blood pressure and relative blood volume change is determined based on the calibration parameter B for particular patient and for every cardiac cycle updated compliance index k. The calculated systolic blood pressure, diastolic blood pressure, and pulse pressure values in the microcontroller are supplied via external communication port.

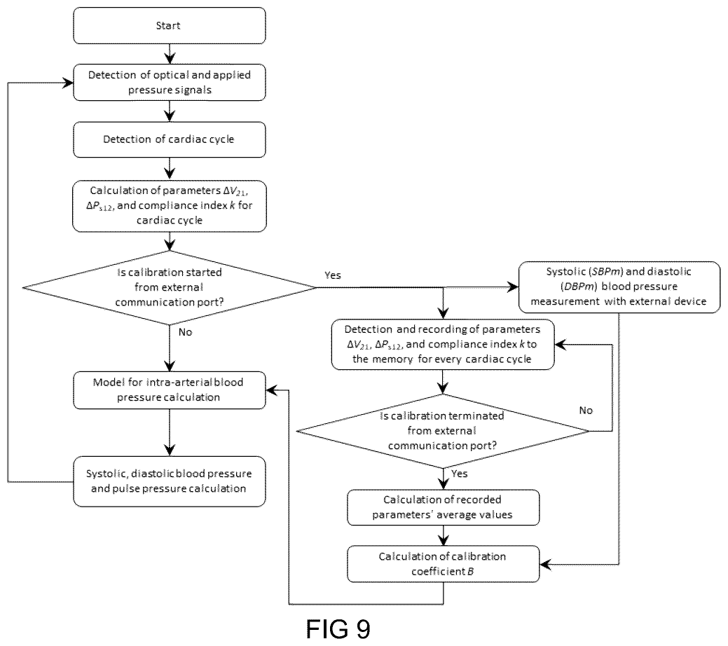

In FIG. 9 is presented flowchart of the method according to present invention with first calibration manoeuvre where: device is attached to the individual and process starts with detection of optical and applied pressure signals, which is followed by detection of cardiac cycle, and based on obtained data parameters ΔV₂₁, Pₛ₁₂, and compliance index k are calculated, which is followed with systolic (SBPₘ) and diastolic (DBPₘ) blood pressure measurement with external device, and detection and recording of parameters ΔV₂₁, Pₛ₁₂, and compliance index k in case calibration is started from external communication port, thereafter detection and recording of parameters is finished in case calibration is terminated from external communication port, which is followed with calculation of recorded parameters' average values and calibration coefficient B, in case calibration is not started or terminated from external communication port it is followed with intra-arterial blood pressure, systolic, diastolic and pulse pressure calculation.

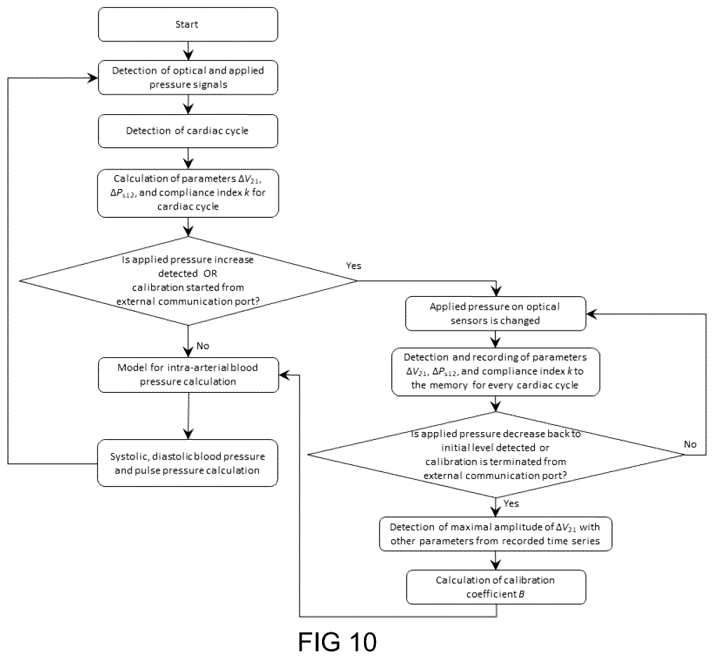

In FIG. 10 is presented flowchart of the method according to present invention with second calibration manoeuvre where: device is attached to the individual and process starts with detection of optical and applied pressure signals, which is followed by detection of cardiac cycle, and based on obtained data parameters ΔV₂₁, Pₛ₁₂, and compliance index k are calculated, which is followed with applied pressure change on optical sensors and detection and recording of parameters ΔV₂₁, Pₛ₁₂, and compliance index k in case applied pressure increase on optical sensors is detected or calibration is started through external communication port, thereafter detection and recording of parameters is finished in case calibration is terminated from external communication port or applied pressure on optical sensors is returned to initial level, which is followed with detection of maximal amplitude of ΔV₂₁ with other parameters from recorded time series and calculation of calibration coefficient B, in case calibration is not started or terminated from external communication port it is followed with intra-arterial blood pressure, systolic, diastolic and pulse pressure calculation.

It is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. For example, sensing light transmitted through rather than back scattered from an artery or microvascular bed of tissue could be utilized to determine relative volume of the artery. Furthermore, any transducer, which converts blood volume or relative blood volume to electrical signal (e.g. bioimpedance), is applicable. The method is not limited with the blood volume measurement sites (e.g. radial artery etc.).

The method according to present invention was tested on three different subjects using two optical sensors, which were attached on the first finger. The applied pressures were different and lower than mean arterial pressure of the finger. The applied pressures were measured and recorded during the experiment. The Finapres system was used for the reference blood pressure measurement. The finger cuff was placed around middle finger. The optical signals were registered with sampling rate of 1 kHz. During the experiment the subject was in supine position. The subjects were asked to carry out hand-grip test in order to change the arterial blood pressure during the recording time. After the recording of the signals the post processing was carried out in MATLAB.

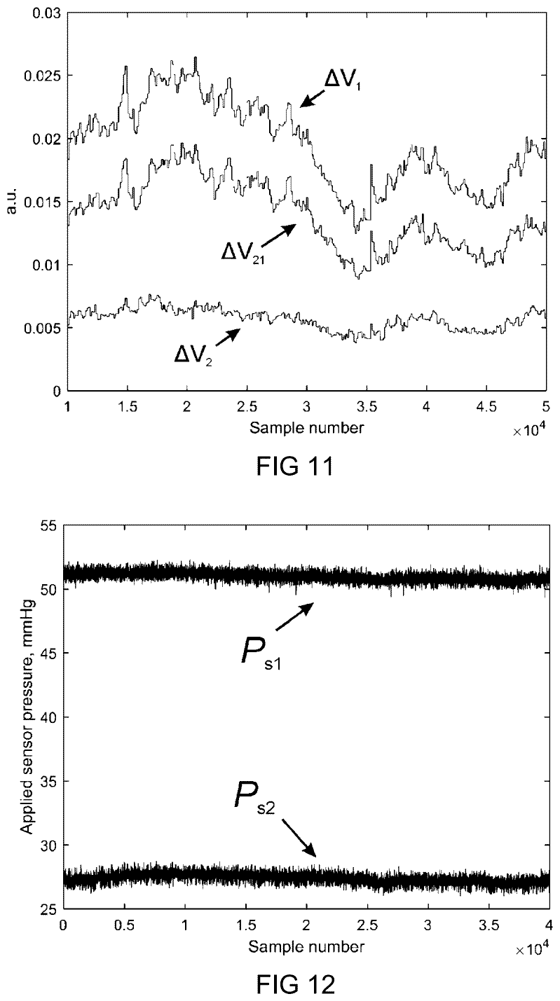

In FIG. 11 are given volumes ΔV₁, ΔV₂, and ΔV₂₁. The recorded pressure signals applied on the sensor are given in FIG. 12. The calibration parameter B was determined using equation 32, according to the measured reference arterial systolic (SBPₘ) and diastolic (DBPₘ) blood pressures.

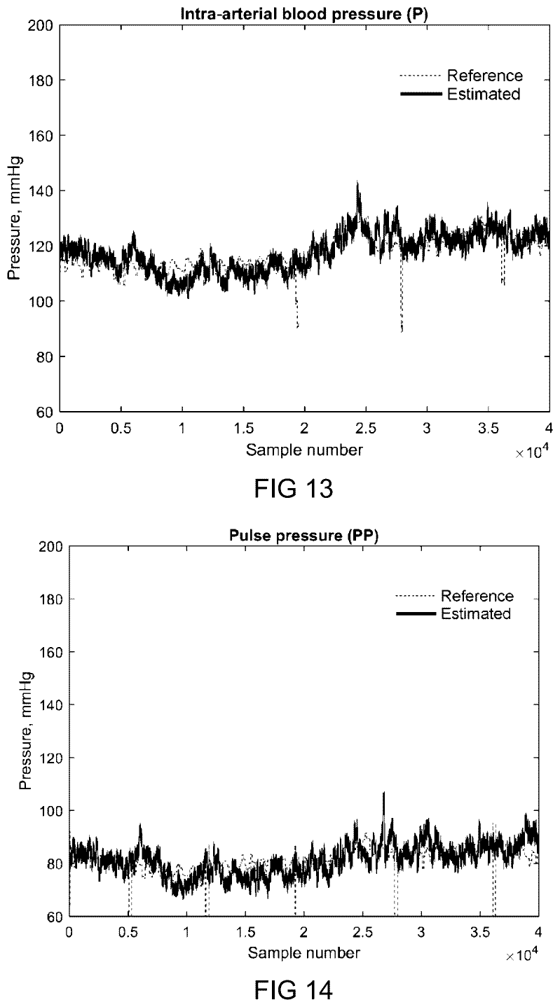

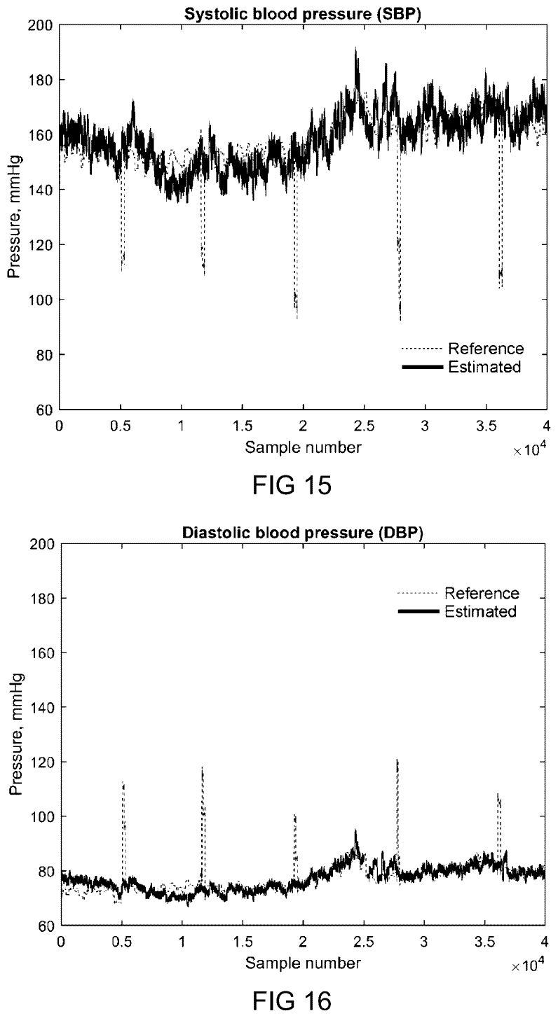

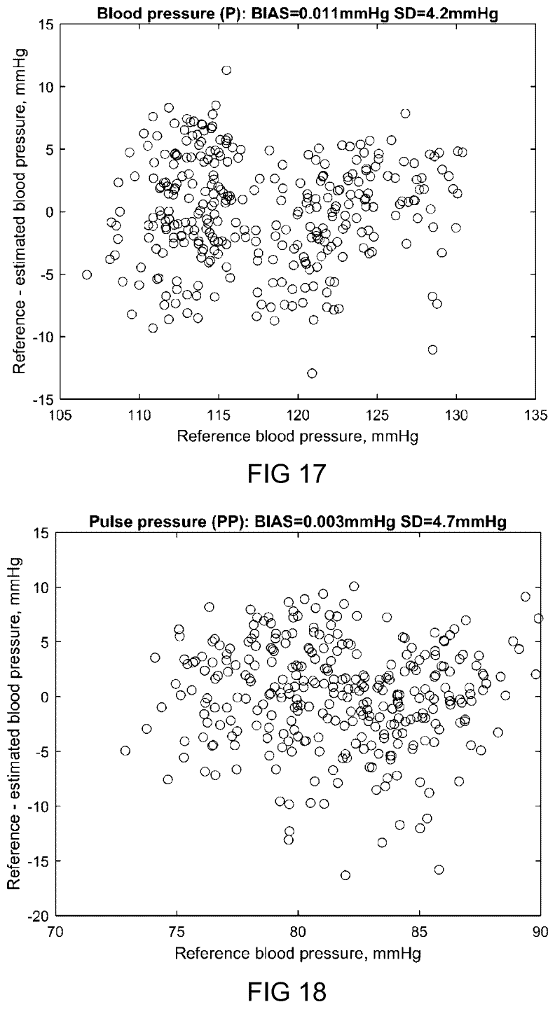

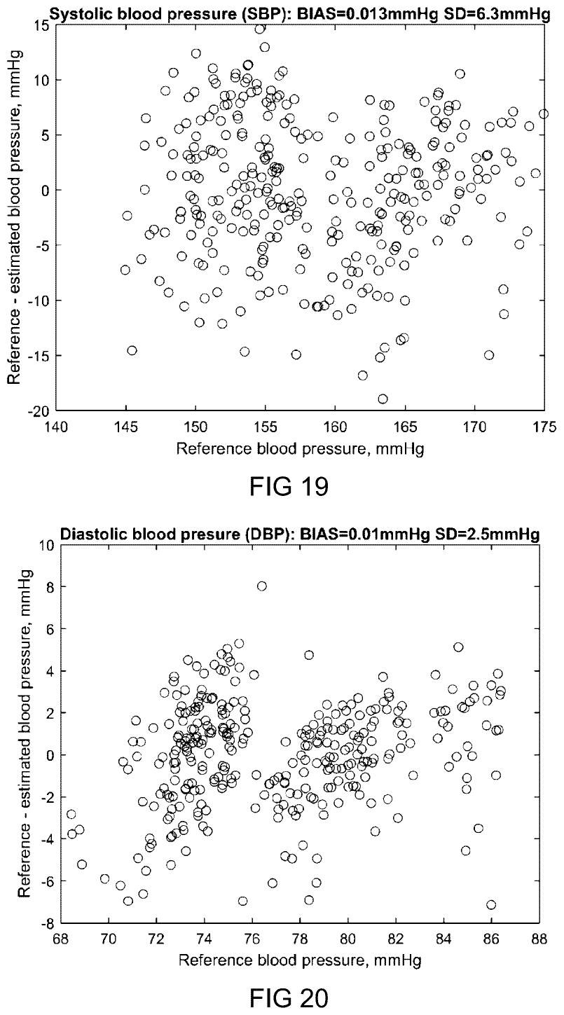

As follows, the blood pressures were estimated using equations 22, and 36 to 39. The results for first subject (subject nr. 1) are illustrated in FIGS. 13 to 16, where the Finapres measured and estimated blood pressures are given. The peaks can be observed from the Finapres measured blood pressure values. Those peaks are related to the sensor calibration of the Finapres device, which occurs after certain period of time. Those Finapres blood pressure values were excluded from the analysis. The Bland-Altman plots of the results are given in FIGS. 17 to 20. The bias (BIAS) and standard deviation (SD) values for each subject and blood pressure are given in Table 1.

TABLE 1
Bias and SD values of the estimated
blood pressures for each subject.

| | | Subject nr. 1 | Subject nr. 2 | Subject nr. 3 |
|---|---|---|---|---|
| SBP | BIAS, mmHg | 0.013 | 0.026 | 0.874 |
| | SD, mmHg | 6.3 | 6.2 | 6.7 |
| DBP | BIAS, mmHg | 0.01 | −0.024 | −1.368 |
| | SD, mmHg | 2.5 | 2.4 | 5.5 |
| PP | BIAS, mmHg | 0.003 | 0.05 | 2.24 |
| | SD, mmHg | 4.7 | 5.8 | 6.2 |
| P | BIAS, mmHg | 0.011 | 0.001 | −0.247 |
| | SD, mmHg | 4.2 | 3.7 | 5.3 |

### LIST OF DETAILS

1, 2—two pairs of photoplethysmographic sensors
3—sensor device housing
4, 5—light source
6, 7—photodetector
8—digital-to-analogue converters (DAC)
9, 10—transimpedance amplifiers
11—analogue-to-digital converters (ADCs)
12, 13—force transducers
14—microcontroller
15—memory
16—external communication port
17—sensor housing
18, 19—optical sensors
20—electrical components of device
21—first spring
22—first optical sensor
23—first force transducer
24—second spring
25—second optical sensor
26—second force transducer
26—subject skin
27—subject artery

1. A device for continuous non-invasive monitoring of arterial blood pressure based on the dependence function of pressure and volume curves for estimating arterial blood pressure, comprising:
two optical sensors consisting of a light source and a photodetector;
digital-analogue converters attached to the light sources;
transimpedance amplifiers electrically connected to the photodetectors;
force transducers attached to the optical sensors;
analogue-to-digital converters electrically connected to the force transducers and transimpedance amplifiers;
a microcontroller electrically connected to the analogue-to-digital converters and digital-to-analogue converters;
a memory electrically connected to the microcontroller; and
an external communication port;
wherein a sensor housing comprises recesses for one or both optocouples in order to produce differences in the back pressures exerted by the optical sensors.

2. A device for continuous non-invasive monitoring of arterial blood pressure based on the dependence function of pressure and volume curves for estimating arterial blood pressure, comprising
two optical sensors consisting of a light source and a photodetector;
digital-to-analogue converters connected to the light sources;
transimpedance amplifiers electrically connected to the photodetectors;
spring loaded force transducers attached to the optical sensors;
analogue-to-digital converters electrically connected to the force transducers and transimpedance amplifiers;
microcontrollers electrically connected to the analogue-to-digital converters and the digital-to-analogue converters;
a memory electrically connected to the microcontroller; and
an external communication port;
wherein a first spring is mounted between the first optical sensor and the first force transducers, the stiffness of which differs 0.1 to 2 times from the stiffness of the second spring mounted between the second optical sensor and the second force transducers, in order to create differences in the back pressures expressed by the optical sensors 2).

3. The device according to claim 1, wherein the difference signal V₁₂ or V₂₁ between the volume curves and amplitude ΔV₁₂ or ΔV₂₁ is calculated in the microcontroller for the determination of arterial blood pressure.

4. The device according to claim 3, wherein the compliance index k of the function between pressure and volume curves is calculated in the microcontroller for each cardiac cycle.

5. The device according to claim 1, wherein the device is automatically switched to calibration mode when an increase in the pressures measured by force transducers is detected or device is switched to the calibration mode through external port, and in which the difference signal amplitude ΔV₁₂ or ΔV₁₂, compliance index k and back pressures Pₛ₁ and Pₛ₂ are stored in the memory attached to the controller for each cardiac cycle and simultaneously their values are sent out through external communication port.

6. The device according to claim 5, wherein the device detects a drop of pressures close to the initial level following an increase in the pressures measured by force transducers, as a result of which the recording of parameters ends or recording is terminated via the external communication port and from the time series of amplitudes ΔV₁₂ the maximum amplitude ΔV_{12_max} and corresponding compliance index k value kₘₐₓ and values of pressures P_{s1_max} ja P_{s2_max} applied by volume sensors are determined and the calibration parameter is calculated.

7. The device according to claim 1, wherein the device is switched to the calibration mode via the external communication port and during which parameters ΔV₂₁ or ΔV₁₂, k, Pₛ₁, Pₛ₂ for each cardiac cycle are stored in the memory attached to the microcontroller.

8. The device according to claim 7, wherein the device is switched off from calibration mode via the external communication port and the systolic (SBPm−) and diastolic (−DBPm−) blood pressure values measured with an external blood pressure device are entered through the said port and based on the time series of the parameters stored in the memory the microcontroller calculates the mean values ΔV_{21_m} ΔV₁₂ m, kₘ, P_{s1_m}. P_{s2_m} after the end of the blood pressure measurement and calculates the calibration parameter B.

9. The device according to claim 6, wherein the arterial blood pressure P, pulse pressure PP, systolic blood pressure SBP and diastolic blood pressure DBP are calculated for each heart cycle in the microcontroller of the device and these values are output via the communication port, respectively.

10. The device according to claim 2, wherein the difference signal V₁₂ or V₂₁ between the volume curves and amplitude ΔV₁₂ or ΔV₂₁ is calculated in the microcontroller for the determination of arterial blood pressure.

11. The device according to claim 2, wherein the device is automatically switched to calibration mode when an increase in the pressures measured by force transducers is detected or device is switched to the calibration mode through external port, and in which the difference signal amplitude ΔV₁₂ or ΔV₁₂, compliance index k and back pressures Pₛ₁ and Pₛ₂ are stored in the memory attached to the controller for each cardiac cycle and simultaneously their values are sent out through external communication port.

12. The device according to claim 11, characterized in that the device detects a drop of pressures close to the initial level following an increase in the pressures measured by force transducers, as a result of which the recording of parameters ends or recording is terminated via the external communication port and from the time series of amplitudes ΔV₁₂ the maximum amplitude ΔV_{12_max} and corresponding compliance index k value kₘₐₓ and values of pressures P_{s1_max} ja P_{s2_max} applied by volume sensors are determined and the calibration parameter is calculated.

13. The device according to claim 2, wherein the device is switched to the calibration mode via the external communication port and during which parameters ΔV₂₁ or ΔV₁₂, k, Pₛ₁, Pₛ₂ for each cardiac cycle are stored in the memory attached to the microcontroller.

14. The device according to claim 13, wherein the device is switched off from calibration mode via the external communication port and the systolic (SBPm) and diastolic (DBPm) blood pressure values measured with an external blood pressure device are entered through the said port and based on the time series of the parameters stored in the memory the microcontroller calculates the mean values ΔV_{21_m} ΔV_{12_m}, kₘ, P_{s1_m}. P_{s2_m} after the end of the blood pressure measurement and calculates the calibration parameter B.

15. The device according to claim 8, wherein the arterial blood pressure P, pulse pressure PP, systolic blood pressure SBP and diastolic blood pressure DBP are calculated for each heart cycle in the microcontroller of the device and these values are output via the communication port, respectively.
